(19) United States
(12) Patent Application Publication
(10) Pub. No.: US 2024/0192198 A1
(43) Pub. Date: Jun. 13, 2024

(54) BIOMIMETIC LIPOSOMES AND METHODS OF MAKING AND USING THE SAME

(71) Applicant: Worcester Polytechnic Institute, Worcester, MA (US)

(72) Inventors: Christina M. Bailey-Hytholt, Shrewsbury, MA (US); Daniel Zimmer, Worcester, MA (US)

(73) Assignee: Worcester Polytechnic Institute, Worcester, MA (US)

(21) Appl. No.: 18/508,058

(22) Filed: Nov. 13, 2023

Related U.S. Application Data
(60) Provisional application No. 63/424,764, filed on Nov. 11, 2022.

(51) Int. Cl. G01N 33/50; B33Y 40/00; B33Y 70/00; G01N 33/531

(52) U.S. Cl. CPC G01N 33/5035; B33Y 70/00; G01N 33/5076; G01N 33/531; B33Y 40/00

(57) ABSTRACT

This disclosure relates generally to biomimetic proteoliposomes, a method of making and a method of using the same. In particular, this disclosure provides proteoliposomes comprising one or more phospholipid carrier and one or more protein embedded in the one or more phospholipid carrier, wherein the one or more phospholipid carrier comprises a phospholipid composition with similar proportions of phospholipids as a naturally occurring cell type and a phospholipid concentration of about 1-50 mM; and wherein the one or more protein comprises a protein composition with similar proportions of proteins as the naturally occurring cell type.

### RELATED APPLICATIONS

This application claims the benefit of and priority to U.S. Provisional Patent Application No. 63/424,764, filed Nov. 11, 2022, which is incorporated herein by reference in its entirety.

### BACKGROUND

Nano-sized vesicles and particles have many different functions in biological systems, such as regulating molecular transport (e.g. small molecules, various proteins, nucleic acids, etc.) and serving as vehicles for communication between cells. Biomimetic systems that recapitulate and are representative of cell membranes can aid in elucidating transport mechanisms at the cellular interface. Additionally, biomimetic systems can be developed to model systems such as extracellular vesicles (EVs), lipoproteins, or viruses by controlling and combining nucleic acid-protein-lipid compositions.

There is a need for highly repeatable and scalable reference standards to allow for accurate and efficient manufacturing of therapeutics and elucidation of transport mechanisms.

### BRIEF SUMMARY

In view of the foregoing, there is a need to develop cell-free and scalable in vitro transport models for high-throughput transport studies of small molecules (e.g. pharmaceuticals, nutrients, toxins, etc.). In some embodiments, such transport models can be used for testing of the ability of small molecules to pass through a placenta. In some embodiments, such transport models can include phospholipid carriers with lipid and protein composition representative of placental trophoblast cells, also noted herein as placental proteoliposomes (PPLs).

In some aspects, the techniques described herein relate to a proteoliposome including: one or more phospholipid carrier and one or more protein embedded in the one or more phospholipid carrier: wherein the one or more phospholipid carrier includes a phospholipid composition with similar proportions of phospholipids as a naturally occurring cell type and a phospholipid concentration of about 1-50 mM; and wherein the one or more protein includes a protein composition with similar proportions of proteins as the naturally occurring cell type.

In some aspects, a ratio of the one or more phospholipid carrier to one or more protein is about 1:100.

In some aspects, the one or more phospholipid carrier includes one or more of phosphatidylcholine (PC), phosphatidylethanolamine (PE), phosphatidylinositol (PI), phosphatidylserine (PS), and sphingomyelin (SPH) and, optionally, cholesterol.

In some aspects, the one or more phospholipid carrier includes a mixture of one or more of PC, PE, PI, PS, and SPH.

In some aspects, the one or more protein includes one or more transmembrane protein.

In some aspects, the one or more transmembrane protein includes one or more of ABCB1, ABCG2, SLC22A5, CD 9, 63, 81, or 82, integrins, Alix, TSG101, clathrin, Ubiquitin, HSP90, HSC90, HSP70, PD-L1, MHC, growth factors, lipoproteins, polymerases, and capsid proteins.

In some aspects, the proteoliposome further includes extracellular matrix (ECM) to form a proteoliposome-ECM composition.

In some aspects, the ECM is formed as a droplet.

In some aspects, the ECM includes one or more of structural proteins, growth factors, and cytokines.

In some aspects, the proteoliposome further includes one or more nucleic acid encapsulated within the proteoliposome.

In some aspects, the one or more nucleic acid does not have modifications to one or more of nucleotides and end capping.

In some aspects, the one or more nucleic acid have modifications to one or more of nucleotides and end capping.

In some aspects, the one or more nucleic acid is an encoding or non-encoding RNA or DNA.

In some aspects, the one or more nucleic acid is present at a concentration of 0-2 mg/mL.

In some aspects, the one or more nucleic acid is present at a concentration of about 0.01-100 μg/mL.

In some aspects, the one or more nucleic acid includes one or more of a chemical bond, nanoparticle, and a conjugation.

In some aspects, the one or more nucleic acid includes RNA, DNA, or a combination thereof,

In some aspects, the RNA includes one or more of mRNA, miRNA, siRNA, and saRNA.

In some aspects, the miRNA includes one or more of 10, 21, 124, 125, 126, 130, and 132.

In some aspects, the techniques described herein relate to a method including: (a) a first mixing of one or more phospholipid to form a phospholipid carrier solution; and (b) a second mixing of the phospholipid carrier solution with a protein solution to produce one or more proteoliposome, wherein the protein solution includes one or more protein.

In some aspects, the second mixing includes a microfluidics approach, wherein the microfluidics approach includes flowing the phospholipid carrier solution and the protein solution through a microfluidic channel under at least one of laminar or turbulent flow.

In some aspects, the second mixing includes an extrusion approach, wherein the extrusion approach includes extruding the phospholipid carrier solution and the protein solution through a porous membrane for a predetermined number of times.

In some aspects, the second mixing includes a combination of a microfluidic approach and an extrusion approach, wherein the microfluidic approach includes flowing the phospholipid carrier solution and the protein solution through a microfluidic channel under at least one of laminar or turbulent flow, and wherein the extrusion approach includes extruding the phospholipid carrier solution and the protein solution through a porous membrane a predetermined number of times.

In some aspects, the phospholipid carrier solution includes ethanol.

In some aspects, the protein solution includes in buffer.

In some aspects, the one or more phospholipid includes one or more of PC, PE, PI, PS, and SPH.

In some aspects, the one or more phospholipid includes a mixture of one or more of PC, PE, PI, PS, and SPH.

In some aspects, the one or more protein includes one or more transmembrane protein.

In some aspects, the one or more transmembrane protein includes one or more of ABCB1, ABCG2, SLC22A5, CD 9, 63, 81, or 82, integrins, Alix, TSG101, clathrin, Ubiquitin, HSP90, HSC90, HSP70, PD-L1, MHC, growth factors, lipoproteins, polymerases, and capsid proteins.

In some aspects, the method further includes a third mixing of each of the one or more proteoliposome with ECM to form one or more proteoliposome-ECM.

In some aspects, the ECM is formed as a droplet.

In some aspects, the ECM includes one or more of structural proteins, growth factors, and cytokines.

In some aspects, the third mixing includes bioprinting.

In some aspects, the bioprinting includes one or more printhead configured for high-throughput.

In some aspects, each of the one or more printhead includes one or more of the one or more proteoliposome and the ECM.

In some aspects, the bioprinting is automated.

In some aspects, the techniques described herein relate to a method for screening pharmaceuticals, including: (a) incubating one or more of a placental proteoliposome (PPL) and a PPL-ECM with one or more molecule of interest to form an incubation product: (b) filtering the incubation product to produce a filtered product; and (c) quantifying the filtered product to assess transport of the one or more molecule of interest into one or more of the PPL and the PPL-ECM.

In some aspects, the one or more molecule of interest includes one or more of a pharmaceutical composition, a nutrient composition and a toxin.

In some aspects, a phospholipid composition of the PPL includes a mixture of one or more of phosphatidylcholine (PC), phosphatidylethanolamine (PE), phosphatidylinositol (PI), phosphatidylserine (PS), and sphingomyelin (SPH) and, optionally, cholesterol.

In some aspects, a protein composition of the PPL includes one or more of ABCB1, ABCG2, and SLC22A5.

### BRIEF DESCRIPTION OF THE DRAWINGS

The novel features of the present disclosure are set forth with particularity in the appended claims. A better understanding of the features and advantages of the present disclosure can be obtained by reference to the following detailed description that sets forth illustrative embodiments, in which the principles of the disclosure are utilized, and the accompanying drawings of which:

FIGS. 1A-1B present schematics for making the proteoliposomes and nucleic acid-loaded proteoliposomes, respectively.

FIG. 2 presents a schematic for bioprinting proteoliposomes into ECM droplets.

FIGS. 3A-3B illustrate models of both proteoliposomes and proteoliposome-ECM droplets, respectively, in the presence of pharmaceuticals or nutrients.

FIGS. 4A-4C present experimental results of PPLs using microfluidic mixing. Three technical replicates are overlaid.

FIGS. 5A-5C present experimental results of PPLs using extrusion. Three technical replicates are overlaid.

FIGS. 6A-6B provide measured absorbance spectra of the PPLs using Folic Acid and Amphotericin B as testing variables, respectively.

FIGS. 7A-7B provide kinetic study results of the PPLS using Folic Acid and Amphotericin B as testing variables, respectively.

FIGS. 8A-8B provide kinetic study results of the PPLS using Folic Acid and Adenosine triphosphate (ATP) showing the activity of the proteins as testing variables, respectively.

FIGS. 9A-9B provide an example of hydrodynamic diameters of PPLs. Three technical replicates are overlaid.

FIGS. 10A-10B present experimental results of isolated EVs in comparison to biomimetic EVs generated by the methods of this disclosure. Three technical replicates are overlaid.

FIG. 11 presents experimental results of nucleic acids that are encapsulated within biomimetic proteoliposomes generated by the methods of this disclosure.

### DETAILED DESCRIPTION

This disclosure relates generally to a composition for mimicking various nanoparticle and membrane systems for use as a reference standard or alternative to the naturally occurring counterpart in analytics and research applications. In some embodiments, the present disclosure provides various biomimetic compositions with the incorporation of lipids and proteins for applications in drug delivery, analytics, cell and animal interaction studies, as well as general industry and research. In some embodiments, this disclosure relates generally to a composition for mimicking active transport in cells, a method of making compositions for mimicking active transport in cells, and a method of using compositions for mimicking active transport in cells.

In some embodiments, this disclosure addresses a need to develop compositions with lipid and protein compositions representative of different cell types for enabling cell-free and scalable in vitro tools for high-throughput transport studies of small molecules. The compositions and methods of the present disclosure may provide additional advantages, such as use for any lipid-protein models for mimicking and assessing active transport of pharmaceutical, nutrient, toxin, or nucleic acid compositions. In some embodiments, the compositions and methods of the present disclosure provide additional advantages such as use for any lipid-protein-nucleic acid models for mimicking cell derived nanoparticles for the use in analytics and research applications.

### Biomimetic Proteoliposomes

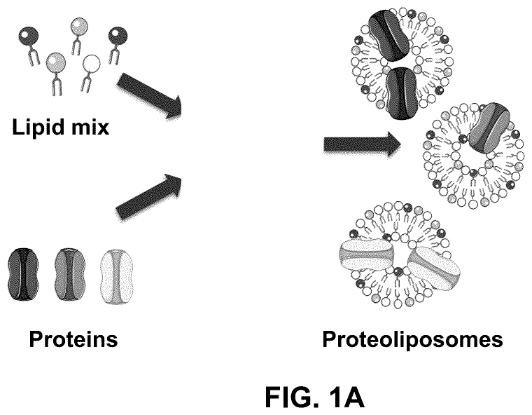

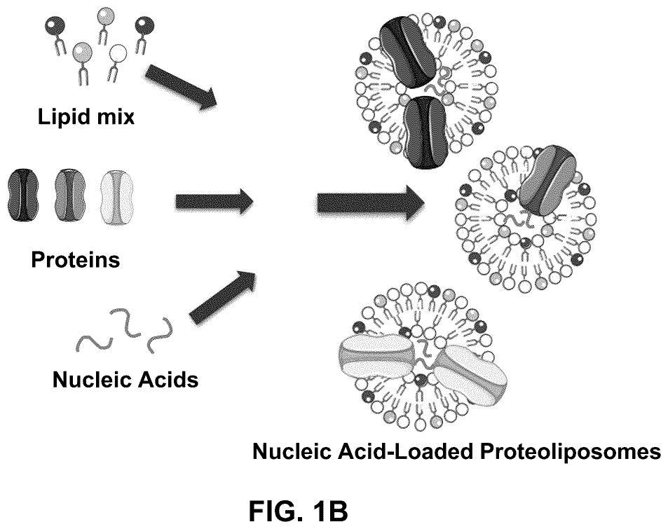

FIGS. 1A-1B illustrate exemplary biomimetic nanoparticles (or proteoliposome) of the present disclosure, either without (FIG. 1A) or with nucleic acids encapsulated within the nanoparticle (FIG. 1B) In some embodiments, the proteoliposome comprises a composition that is biomimetic of various model systems. Proteoliposomes are examples of nanoparticles that include of a lipid bilayer with an integrated transmembrane protein. The incorporation of lipids, proteins, and/or nucleic acids with tuned parameters enables the development of model lipid membranes and vesicles with applications where only specific components of the full biological system are needed.

Biomimetic proteoliposomes representative of membranes of cells can aid in elucidating transport mechanisms at the cellular interface, and may provide highly useful tools for enabling rapid screening studies at effective costs. In some embodiments, the biomimetic proteoliposomes can be used to model nanoparticle systems such as liposomes, lipid membranes, extracellular vesicles (EVs), endosomes, lipoproteins, or viruses with combinations of nucleic acid-protein-lipid biomimetics. In some embodiments, the proteoliposomes of this disclosure may have different structures including hollow proteoliposomes, i.e., vesicles, or proteoliposomes with solid cores.

In some embodiments, as illustrated in FIG. 1A, the biomimetic proteoliposomes comprise one or more phospholipid carrier and one or more proteins embedded in the one or more phospholipid carrier.

In some embodiments, the phospholipid carrier of the present disclosure may comprise a lipid bilayer. The lipid bilayer in various nanoparticle systems has many different functions in biological systems, such as regulating drug transport and providing structure for transmembrane proteins. In some embodiments, the lipid bilayer is a planar bilayer.

In some embodiments, the one or more phospholipid carrier includes one or more phospholipids. In some embodiments, the phospholipids of the same type may form the phospholipid carrier. In some embodiments, the phospholipid carrier is formed by a mixture of different phospholipids. In some embodiments, the biomimetic phospholipid carriers are produced from the mixture of different lipid species in similar proportions as a naturally occurring cell type. In some embodiments, the one or more phospholipid are synthetically generated or extracted from cells so as to produce a biomimetic phospholipid carrier or composition.

In some embodiments, the one or more phospholipid may be naturally occurring or synthetic phospholipids. For examples, the one or more phospholipids may include, but not limited to, one or more naturally occurring phospholipids such as phosphatidylcholine (PC), phosphatidylethanolamine (PE), phosphatidylinositol (PI), phosphatidylserine (PS), and sphingomyelin (SPH), phospatidylglycerol (PG), phosphatidic acids (PA) or PEGylated phospholipids. In some embodiments, such phospholipids may be synthetically produced to mimic the naturally occurring phospholipids. In some embodiments, the one or more phospholipid may be further modified to have various desired functions. In some embodiments, for example, a PEGylated phospholipid may function to add steric bulk. In some embodiments, for example, a fluorescent phospholipid may allow for imaging or more in-depth analysis of the phospholipid carrier. In some embodiments, for example, an ionizable lipid may function to incorporate an nucleic acid. In some embodiments, the synthetically generated phospholipid may further be conjugated to one or more of a protein or peptide.

In some embodiments, the one or more proteins embedded in the proteoliposomes of the present disclosure may comprise multiple proteins, of the same type or different types. In some embodiments, the one or more protein comprises one or more transmembrane protein. The type of transmembrane protein may vary based on the type of nanoparticle system to be modeled. In some embodiments, the one or more transmembrane protein may include ß-sheet transmembrane proteins and a-helical transmembrane proteins. In some embodiments, the transmembrane protein is a G-protein coupled receptor (GPCR), an ion channel, a transporter, a glycophorin, an integrin, a cadherin, a selectin, a cluster of differentiation (CD) protein or a porin. In some embodiments, the GPCR is a metabotropic receptor or a non ligand-mediated GPCR. In some embodiments, ion channel proteins include potassium channel proteins, TRP channel proteins, four-domain channel proteins, chloride channel proteins, ligand-gated ion channel proteins, transporter proteins. CD marker proteins, tetraspannin proteins, and aquaporin proteins. In some embodiments, the transmembrane proteins include VIPRI, ADORA2A, F2R, EP4, CXCR4, LPAR1, GRPR, ADRB2, EAG, Navl 0.7, CLCA1, nAChR, ABCA1, SLC5A1, MS4A1, AQP1, CD33, CD20, DARC, Kir2.1, Kir2.2, Kir7.1. KvIO.1, Kvl 1. 1, TASK3, TRPV3, and TRPV3.

In some embodiments, the transmembrane protein may be a channel protein so as to be biomimetic of the lipid membrane of a particular cell type, A channel protein is a pore-forming membrane protein, which may allow for the transport of molecules such as ions and small molecules across a cell membrane. In some embodiments, the one or more channel protein is a ligand-gated, voltage-gated, or mechanically-gated channel protein. In some embodiments, the one or more channel protein allows for passive diffusion of molecules such as ions and small molecules. In some embodiments, the one or more channel protein comprises one or more channel proteins of the ATP binding cassette (ABC), solute carrier (SLC) family or both. In some embodiments, the one or more channel protein of the ABC and SLC family comprises ABCB1, ABCG2, or SLC22A5. In some embodiments, additionally or alternatively, the one or more transmembrane proteins may comprise tetraspannin proteins—such as CD proteins including CD 9, 63, 81, or 82—integrins, Alix, TSG101, clathrin, Ubiquitin, HSP90, HSC90, HSP70, PD-L1, MHC, growth factors, lipoproteins, and polymerases. In some embodiments, the one or more channel protein may include, but not limited to, VDAC1 Voltage-dependent anion-selective channel protein, VDAC2 Voltage-dependent anion-selective channel protein, and VDAC3 Voltage-dependent anion-selective channel protein,

In some embodiments, the one or more proteins comprise capsid proteins. In some embodiments, the one or more proteins comprise carrier proteins, Carrier proteins are integral proteins that transport chemicals across the membrane both down and up the concentration gradient.

In some embodiments, as illustrated in FIG. 1B, the proteoliposomes of the present disclosure may comprise one or more phospholipid carrier and one or more proteins embedded in the one or more phospholipid carrier, as discussed in connection with FIG. 1A above, and may further comprise one or more nucleic acid encapsulated within the one or more phospholipid carrier. In some embodiments, the one or more nucleic acid is an RNA (such as mRNA, miRNA, siRNA, saRNA, or other form of RNA) or DNA with or without modifications to nucleotides and/or end capping. The type of nucleic acid loaded into the proteoliposome may vary based on the type of assay to be performed. For example, a transfection study may be used to assess mRNA transport, and/or a scratch test may be used to assess miRNA or siRNA transport. In some embodiments, a proteoliposome loaded with saRNA may be assessed for efficacy in mimicking a virus. In some embodiments, the one or more nucleic acid is an encoding or non-encoding RNA or DNA, Examples of the one or more nucleic acid include miRNA 10, 21, 124, 125, 126, 130, and/or 132, In some embodiments, the one or more nucleic acid is present at a concentration of 0-2 mg/mL. In some embodiments, the one or more nucleic acid is present at a concentration of about 0.01-100 μg/mL. In some embodiments, the nucleic acid utilizes a chemical bond, nanoparticle, or conjugation to increase an encapsulation of the nucleic acid.

In some embodiments, the composition of the proteoliposomes are designed to mimic transport through a particular cell membrane or to mimic EVs of various cells. In some embodiments, the cells being mimicked are pluripotent stem cells, adult stem cells, mesenchymal stem cells, embryonic stem cells, cardiovascular stem cells (CDCs), placental stem cells, induced pluripotent stem cells and other stem cells such as somatic stem cells, hematopoietic stem cells, neural stem cells, osteoblasts, cancer stem cells, epithelial stem cells, and bone marrow stem cells. In some embodiments, the cells being mimicked are kidney cells, cancer cells, epidermal cells, immune cells, placental cells, mucosal cells, fibroblasts, blood brain barrier cells and other cells.

In some embodiments, the composition of the proteoliposomes are designed to mimic transport into placental cells. In some embodiments, the phospholipids of the proteoliposomes are selected such that the composition of the phospholipid carrier mimics the phospholipid composition of the placenta. For the purposes of the present disclosure, a placental proteoliposome may be referred to as PPL. In some embodiments, the composition of phospholipids in the PPL comprises a percentage of composition of about 40-60% PC, 10-35% PE, 1-20% PI, 1-20% PS, and 1-25% SPH. In some embodiments, the composition of phospholipids in the PPL comprises a percentage of composition of about 50.7±0.7% PC, 23.3±0.6% PE. 7.0±1.6% PI, 9.1±0.9% PS, and 11.2±0.7% SPH. In some embodiments, the cells being mimicked are any cell residing in the placenta. In some embodiments, the placental cells are placental trophoblast cells. The one or more transmembrane protein of the proteoliposome may comprise a channel protein commonly found in the placenta. In some embodiments, the one or more channel protein comprises one or more channel proteins of the ATP binding cassette (ABC), solute carrier (SLC) family or both. In some embodiments, the one or more channel protein of the ABC and SLC family comprises ABCB1, ABCG2, or SLC22A5.

In some embodiments, the proteoliposomes of this disclosure are biomimetic of an EV of the particular cell types described herein. For example, many EV-based therapeutics use stem cells which are cultured continuously, with EVs being extracted from the culture media. During this culturing the exposure to growth factors and other variables may affect the differentiation and thus the therapeutic efficacy of the derived therapeutics. Because of the altering expression of the cells there is a need for a highly repeatable reference standard to allow for accurate and efficient manufacturing of therapeutics. The proteoliposomes of this disclosure may meet this need by providing scalable and customizable models that are biomimetic of each of a variety of EVs. In some embodiments, the one or more proteins may comprise tetraspannins proteins—such as CD proteins including CD 9, 63, 81, or 82-integrins, Alix, TSG101, clathrin, Ubiquitin, HSP90, HSC90, HSP70, PD-L1, MHC, growth factors, lipoproteins, and polymerases, so as to be biomimetic of the EVs. In some embodiments, the proteoliposomes that are biomimetic of EVs may comprise any of the aforementioned proteins.

In some embodiments, the composition of the proteoliposomes is designed to mimic transport through a virus. In some embodiments, the phospholipids of the proteoliposomes are selected such that the composition of the phospholipid carrier mimics the phospholipid composition of a virus. In some embodiments, the one or more proteins of the proteoliposomes that are biomimetic of a virus comprises capsid proteins. In some embodiments, the proteoliposomes that are biomimetic of viruses further comprise one or more nucleic acid encapsulated within the one or more phospholipid carrier. The one or more nucleic acid encapsulated within the proteoliposome may be an RNA (such as mRNA, miRNA, siRNA, saRNA, or other form of RNA) or DNA with or without modifications to nucleotides and/or end capping.

### Methods of Making Proteoliposomes

In some embodiments, the present disclosure provides methods of making proteoliposomes for mimicking active transport in a model cell or nanoparticle system. The methods of this disclosure may be used for obtaining proteoliposomes with greater efficiency and control over previous methods, allow for commercialization and scale-up. In some embodiments, this disclosure employs a microfluidic and/or extrusion-based mixing platform that may be utilized to produce proteoliposomes with uniform size distributions, low polydispersity, and batch to batch reproducibility.

In some embodiments, the methods of the present disclosure may produce proteoliposomes by mixing one or more solutions. In some embodiments, the solutions may be mixed using microfluidic techniques, extrusion techniques, or both. Each of the first solution and the second solution may comprise an organic phase solution, an aqueous phase solution, or a combination thereof. For example, the first solution may be an organic phase solution and the second solution may be an aqueous phase solution. In some embodiments, the organic phase solution may include the one or more phospholipids, the one or more protein or both in a volatile liquid, such as, for example, ethanol. In some embodiments, the aqueous phase solution may include the one or more phospholipids, the one or more protein or both in a buffer or saline. In some embodiments, the one or more phospholipids may be a phospholipid mixture of multiple types of phospholipids. In some embodiments, the mixing of the phospholipid mixture with the one or more proteins produce the proteoliposomes.

In some embodiments, the proteoliposomes may be formed by mixing an organic phase solution including the one or more phospholipid with an aqueous phase solution comprising the one or more proteins. In some embodiments, the organic phase solution may also include one or more proteins, which may be the same or different than the proteins in the aqueous phase. The addition of proteins into the organic phase solution may more reliably integrate the protein into the resulting phospholipid bilayer. If protein is only added with the aqueous phase, this may increase the concentration of protein within the phospholipid bilayer, but may affect the protein folding and thus provide less control over the integration of the protein within the lipid bilayer.

As indicated above, the methods of mixing the first and second solutions may comprise a microfluidic approach, an extrusion approach, or a combination thereof for manufacturing the proteoliposomes. For example, the method of making proteoliposomes may comprise using a microfluidic approach followed by an extrusion approach, a microfluidic approach without an extrusion approach, or an extrusion approach without a microfluidic approach.

The microfluidics approach for mixing the solutions comprises flowing one or more of the first solution and the second solution through a microfluidic channel under laminar or turbulent flow. In some embodiments, the microfluidic approach is performed using flow rates of each solution, e.g., an organic phase flow rate and an aqueous phase flow rate, from 0.2 mL/min to 20 mL/min. In some embodiments, the organic phase flow rate and aqueous phase flow rate flow are at a predetermined ratio. In some embodiments, the predetermined ratio correlates to Reynolds numbers of near zero to the turbulent regime.

The extrusion approach comprises mixing one or more of the first solution and the second solution, and extruding the mixture through a membrane. For example, the mixing in the extrusion approach may comprise forming an organic phase solution of a mixture of one or more phospholipids and/or proteins, applying gas to the mixture (e.g., nitrogen) to form a thin film, vacuuming the thin film to remove the organic phase, rehydrating with a buffer solution, and further mixing the rehydrated solution with agitation such as, for example, by vortexing. This may form larger vesicles in the mixture that may be extruded through the membrane. In some embodiments, the membrane is porous, such that extrusion of the mixture through the membrane forms proteoliposomes allows for control over the sizing and uniformity of the proteoliposomes. In some embodiments, a pore size of the membrane may range from about 1 nm to about 1 mm. The mixture of proteoliposomes may be extruded through the membrane any number of times to achieve a desired uniformity. In some embodiments, the resulting proteoliposomes are diluted any number of times to achieve a desired concentration. In some embodiments, the resulting proteoliposomes are stabilized by using a buffer exchange.

In some embodiments, the proteoliposomes will go through an additional extrusion or filtration with a bilayer which may or may not contain one or more protein. Additional extrusion or filtration with a bilayer containing one or more protein may provide further incorporation of the one or more protein into the proteoliposome, whereas without the protein will yield no further addition of protein into the proteoliposome.

In some embodiments, the mixing is performed using a range of ratios of aqueous phase solution to organic phase solution from 1:1 and beyond. In some embodiments, the aqueous phase solution is acidic. In some embodiments, the aqueous phase solution is basic. In some embodiments, the aqueous phase solution is polar. In some embodiments, the organic phase solution is configured to dissolve the one or more phospholipid. In some embodiments, a ratio of the one or more phospholipid to one or more protein is 1:100. In some embodiments, a concentration of the one or more phospholipid is 1-50 mg/mL. In some embodiments, a concentration of the one or more phospholipid is 1-50 mM. In some embodiments, the proteoliposomes can either be concentrated or diluted to meet the desired concentration.

Depending on the model system, the method of making will vary in the use of the particular phospholipid or protein composition to be used. For example, each of the one or more phospholipid includes one or more of phosphatidylcholine (PC), phosphatidylethanolamine (PE), phosphatidylinositol (PI), phosphatidylserine (PS), and sphingomyelin (SPH), or cholesterol. In some embodiments, the one or more phospholipid are synthetically generated or extracted from the target cells or cell membrane to be mimicked so as to produce a biomimetic phospholipid carrier specific to that cell type.

In some embodiments, the one or more proteins to be mixed may be one or more channel proteins of the ATP binding cassette (ABC), solute carrier (SLC) family or both, such as, for example, ABCB1, ABCG2, or SLC22A5. In some embodiments, the one or more proteins to be mixed may be tetraspannins—e.g., CD 9, 63, 81, or 82—integrins, Alix, TSG101, clathrin, Ubiquitin, HSP90, HSC90, HSP70, PD-L1, MHC, growth factors, lipoproteins, and/or polymerases, so as to mimic EVs.

In some embodiments, the method of making the proteoliposomes may be modified for loading the proteoliposomes with nucleic acids. In some embodiments, the phospholipid mixture is formed by an organic phase solution containing the one or more phospholipid in ethanol, with or without the one or more protein, mixed with an aqueous phase solution containing buffer, with the one or more protein and the one or more nucleic acid, thereby forming nucleic acid-loaded proteoliposomes. Similarly, the phospholipid mixture may be formed by an organic phase solution containing the phospholipids with proteins in ethanol, mixed with an aqueous solution containing the proteins and the nucleic acids in the buffer. In some embodiments, the phospholipid mixture may be formed by an organic phase solution containing the phospholipids in ethanol, without the proteins, mixed with an aqueous solution containing the proteins and the nucleic acids in the buffer. Concentrations of nucleic acids or proteins can be increased or decreased through concentration within the proteoliposomes or the concentration of the proteoliposomes in the solution.

In some embodiments, the one or more nucleic acid is an RNA (such as mRNA, miRNA, siRNA, saRNA, or other form of RNA) or DNA with or without modifications to nucleotides and/or end capping. In some embodiments, the one or more nucleic acid is an encoding or non-encoding RNA or DNA. Examples of the one or more nucleic acid include miRNA 10, 21, 124, 125, 126, 130, and/or 132. In some embodiments, the one or more nucleic acid is present at a concentration of 0-2 mg/mL. In some embodiments, the one or more nucleic acid is present at a concentration of about 0.01-100 μg/mL. In some embodiments, the nucleic acid utilizes a chemical bond, nanoparticle, or conjugation to increase an encapsulation of the nucleic acid.

In some embodiments, the present disclosure provides methods of making placental proteoliposomes (PPLs). In some embodiments, the organic phase solution includes the one or more phospholipid placental lipid components in ethanol and the aqueous phase solution includes the one or more placental channel protein in a buffer. In some embodiments, this mixing is performed using a range of ratios of aqueous phase solution to organic phase solution from 1:1 and beyond. In some embodiments, the aqueous phase solution is polar. In some embodiments, the organic phase solution is configured to dissolve the one or more phospholipid. In some embodiments, the microfluidic mixing is performed using flow rates of each solution. e.g., an organic phase flow rate and an aqueous phase flow rate, from 0.2 mL/min to 20 mL/min. In some embodiments, the organic phase flow rate and aqueous phase flow rate flow at a predetermined ratio. In some embodiments, the predetermined ratio correlates to Reynolds numbers of near zero to the turbulent regime. In some embodiments, a ratio of the one or more phospholipid to one or more protein is 1:100. In some embodiments, a concentration of the one or more phospholipid is 1-50 mg/mL. In some embodiments, a concentration of the one or more phospholipid is 1-50 mM. In some embodiments, the proteoliposomes can either be concentrated or diluted to meet the desired concentration.

### Methods of Making Proteoliposome-ECM

In some embodiments, the present disclosure provides a composition of one or more of the proteoliposomes provided herein within a medium. In some embodiments, the medium comprises extracellular matrix (ECM). In some embodiments, the medium comprises collagen. In some embodiments, the medium comprises agar. In some embodiments, the medium comprises alginate. In some embodiments, the medium is in the shape of a cell- or tissue-representative droplet. In some embodiments, the droplet is representative of placental cell or tissue.

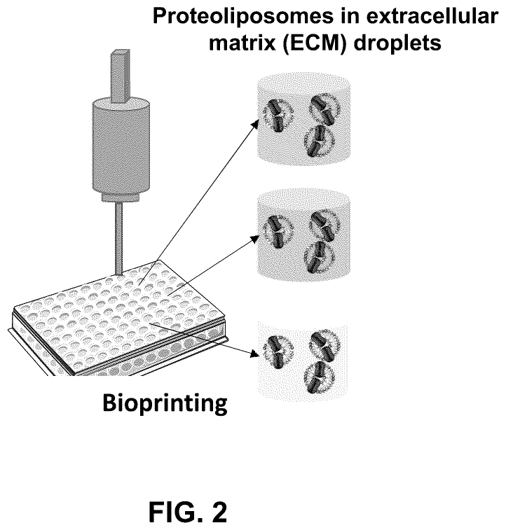

FIG. 2 presents a schematic for bioprinting the cell-free model to form a proteoliposome-ECM. In some embodiments, the cell-free model comprises proteoliposomes bioprinted into a medium. In some embodiments, the medium comprises ECM. In some embodiments, the ECM comprises one or more of collagen, laminin, and fibronectin. In some embodiments, the medium comprises ECM droplets. In some embodiments, the ECM is configured to cross-link. In some embodiments, the ECM is bioprinted as a droplet. In some embodiments, bioprinting comprises multiple printheads configured for high-throughput. In some embodiments, each of the multiple printheads comprises one or more of the NPs and the ECM. In some embodiments, the bioprinting is automated. In some embodiments, the ECM comprises one or more of structural proteins, growth factors, and cytokines. In some embodiments, one or more PPL is bioprinted onto ECM to form a PPL-ECM.

### Methods of Using Proteoliposomes and Proteoliposome-ECMs

The compositions and methods of the present disclosure relates to the development of proteoliposomes, in the absence of cells, to facilitate active transport screening of a wide range of pharmaceuticals by incorporating key transmembrane proteins that have been identified as important components for various cell transport mechanisms. These methods further allow for the understanding of how pharmaceuticals impact nutrient transport.

In particular, the compositions and methods of the present disclosure may be used for any proteoliposome or nucleic-acid loaded proteoliposome models for mimicking cell-derived nanoparticles in analytics and research applications. For example, the proteoliposomes disclosed herein can be used as reference standards for analytical assays that provide concentrations or critical quality attributes of products. Such assays may be used, as provided herein, for evaluating an efficacy and function of the biomimetic proteoliposomes of this disclosure in comparison to cells or cell-derived nanoparticles, depending on the type of cell or nanoparticle. These quality attributes can be assessed through cell- or animal-based assays, such as, for example, scratch tests or other wound healing studies, or other transfection efficiency studies. Additional assays that such proteoliposomes may be applied include chemical assays such as immunoassays. Bicinchoninic acid (BCA) assays, or fluorescence-based nucleic acid quantitation assays, as well as PCR-based analytics such as capillary electrophoresis, mass spectrometry, chromatography, or SDS-page.

Further, transport of nutrients and pharmaceuticals may be impacted by the extracellular matrix (ECM) composition of a particular cell or tissue. In order to investigate how the ECM impacts transport with the proteoliposomes, the proteoliposomes are bioprinted into cell- or tissue-representative ECM droplets. Again, this technology allows for automation and scale up in order for the proteoliposomes and proteoliposome-ECM compositions to be commercially translatable. This method enables the screening of transport of pharmaceuticals and nutrients using the proteoliposome-ECM droplet models.

The advantages of this technique are: cell-free allowing for a facile, easy to use, high-throughput screening: scale-up ready technology (microfluidic and/or extrusion mixing and bioprinting technologies) employed to formulate the proteoliposomes and the proteoliposome-ECM droplets, allowing for the ability to automate the manufacturing process; and ability to incorporate cell- or tissue-representative lipids, proteins, and ECM. Design of experiments (DOE) can be utilized to parse out interactions due to each component.

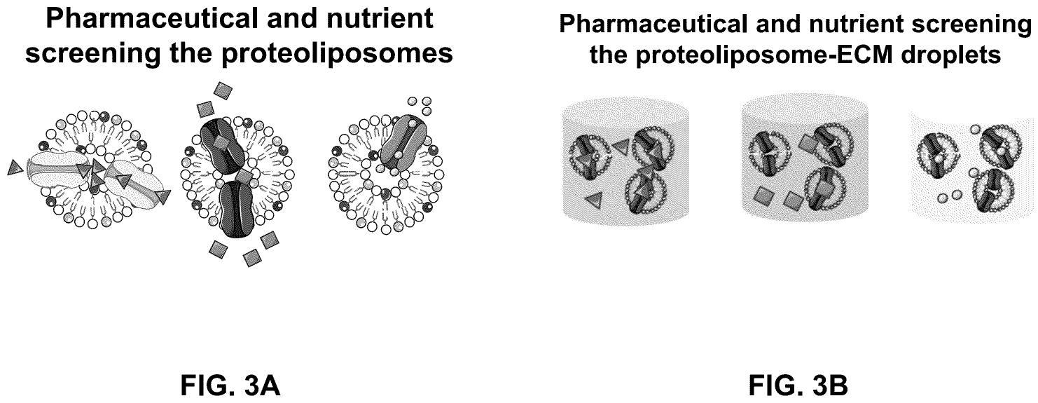

FIGS. 3A-3B illustrate models of the proteoliposome and the proteoliposome-ECM for use in pharmaceutical and nutrient screening. In some embodiments, the proteoliposome for use in pharmaceutical and nutrient screening may comprise biomimetic proteoliposomes tailored to mimic particular cell or cell membranes. EVs. or viruses, based on the phospholipid, protein, and/or nucleic acid composition used to generate the proteoliposome. In some embodiments, the compositions comprise models of both proteoliposomes and proteoliposome-ECM, respectively, in the presence of molecules of interest. In some embodiments, the method of using proteoliposomes for mimicking active transport in cells comprises assessing the one or more proteoliposomes with one or more molecule of interest.

In some embodiments, the method of using proteoliposomes for mimicking active transport in cells comprises assessing the one or more proteoliposome-ECM. In some embodiments, the one or more molecule of interest comprises one or more of a pharmaceutical composition and a nutrient composition. In some embodiments, the assessing comprises incubating one or more of the one or more proteoliposomes and one or more proteoliposomes-ECM with the one or more molecule of interest to produce an incubation product. In some embodiments, the assessing comprises filtering the incubation product to produce a filtered product. In some embodiments, the filtered product is quantified to measure transport of the one or more molecule of interest into one or more of the one or more proteoliposomes and one or more proteoliposomes-ECM. In some embodiments, the proteoliposomes or proteoliposome-ECM assessed comprise PPLs or PPL-ECM.

### Additional Embodiments

In some aspects, the techniques described herein relate to a composition including: one or more transport model, wherein the one or more transport model includes one or more phospholipid carrier and one or more protein embedded in the phospholipid carrier: wherein the one or more phospholipid carrier includes at least one of a lipid bilayer and a liposome.

In some aspects, the one or more phospholipid carrier includes one or more of phosphatidylcholine (PC), phosphatidylethanolamine (PE), phosphatidylinositol (PI), phosphatidylserine (PS), and sphingomyelin (SPH). In some aspects, the one or more phospholipid carrier may further include cholesterol.

In some aspects, the one or more phospholipid carrier includes a mixture of one or more of PC, PE, PI, PS, and SPH.

In some aspects, the one or more protein includes one or more transmembrane protein.

In some aspects, the one or more transmembrane protein includes one or more of ABC or SLC transporters (such as ABCB1, ABCG2, and SLC22A5), tetraspannins (such as CD 9, 63, or 81), capsid protein or lipoprotein.

In some aspects, the one or more nucleic acid is incorporated into the mixture of proteins and/or lipids.

In some aspects, the composition further includes extracellular matrix (ECM) to form a PPL-ECM.

In some aspects, the ECM is formed as a droplet.

In some aspects, the ECM includes one or more of structural proteins, growth factors, and cytokines.

In some aspects, the techniques described herein relate to a method including: (a) a first mixing of one or more phospholipid to form a phospholipid solution; and (b) a second mixing of the phospholipid solution with a protein solution to produce one or more phospholipid carrier, wherein the protein solution includes one or more protein.

In some aspects, the second mixing includes a microfluidics approach, wherein the microfluidics approach includes flowing the phospholipid solution and the protein solution through a microfluidic channel under at least one of laminar or turbulent flow.

In some aspects, the second mixing includes an extrusion approach.

In some aspects, the phospholipid solution includes ethanol.

In some aspects, the protein solution includes in buffer.

In some aspects, the one or more phospholipid includes one or more of PC, PE, PI, PS, and SPH.

In some aspects, the one or more phospholipid includes a mixture of one or more of PC, PE, PI, PS, and SPH.

In some aspects, the one or more protein includes one or more transmembrane protein.

In some aspects, the one or more transmembrane protein includes one or more of ABCB1, ABCG2, and SLC22A5.

In some aspects, the method further includes a third mixing of each of the one or more phospholipid carrier with ECM to form one or more carrier-ECM.

In some aspects, the ECM is formed as a droplet.

In some aspects, the ECM includes one or more of structural proteins, growth factors, and cytokines.

In some aspects, the third mixing includes bioprinting.

In some aspects, the bioprinting includes one or more printhead configured for high-throughput.

In some aspects, each of the one or more printhead includes one or more of the one or more phospholipid carrier and the ECM.

In some aspects, the bioprinting is automated.

In some aspects, the techniques described herein relate to a method for screening pharmaceuticals, including: (a) incubating one or more of a phospholipid carrier and carrier-ECM with one or more molecule of interest to form an incubation product: (b) filtering the incubation product to produce a filtered product; and (c) quantifying the filtered product to assess transport of the one or more molecule of interest into one or more of the phospholipid carrier and carrier-ECM.

In some aspects, the one or more molecule of interest includes one or more of a pharmaceutical composition, a nutrient composition and a toxin.

In some aspects, the nucleic acid utilizes a chemical bond, nanoparticle, or conjugation to increase the binding, entrapment, and/or encapsulation of the nucleic acid.

In some aspects, the nucleic acid is an RNA (such as mRNA, miRNA, siRNA, saRNA, or other form of RNA) or DNA with or without modifications to nucleotides and/or end capping.

In some aspects, the overall particle is to mimic membranes and vesicles, such as extracellular vesicles (EVs), endosomes, lipoproteins, or viruses.

In some aspects, the composition includes a nucleic acid, protein, or lipid is developed to be representative of the naturally occurring counterpart.

### Exemplary Embodiments

While preferred embodiments of the present disclosure have been shown and described herein, it will be obvious to those skilled in the art that such embodiments are provided by way of example only. Numerous variations, changes, and substitutions will now occur to those skilled in the art without departing from the present disclosure. It should be understood that various alternatives to the embodiments described herein, or combinations of one or more of these embodiments or aspects described therein may be employed in practicing the present disclosure. It is intended that the following claims define the scope of the present disclosure and that methods and structures within the scope of these claims and their equivalents be covered thereby.

### Example 1: Placental Proteoliposome Compositions

Currently, less than 10% of U.S. Food and Drug Administration (FDA) approved pharmaceuticals have enough information to determine risks towards a developing fetus. However. 9 out of 10 women in the U.S. take one or more medication during the course of their pregnancy. The Centers for Disease Control and Prevention (CDC) has identified this issue and actively has a Treating for Two program to help identify safe treatment options to improve the health of women and babies. In order to facilitate this need, the development of new in vitro tools to assess how molecules and macromolecules, such as pharmaceuticals, interact with the maternal-fetal interface is crucial, as are standardized models by which such tools are compared for efficacy. Biomimetic systems representative of the placenta can aid in elucidating transport mechanisms at the maternal-fetal interface.

The placenta plays an important role during pregnancy, yet it remains one of the least understood human organs. The main cell type composing the placenta, trophoblast cells, have important functions including nutrient and waste transport, invading the endometrium to anchor the placenta, and remodeling vasculature for adequate blood flow. These placental trophoblast cells have great potential for use in developing in vitro models of the maternal-fetal interface. Current models for understanding placental transport are extremely limited. The placenta is the most species-specific organ, which creates challenges for assessing in vivo studies. Due to this challenge, emerging technologies, including placental microtissues and placenta-on-a-chip microfluidic devices are being studied. However, so far there are no commercially available cell-free models of the placenta to provide highly useful tools for enabling rapid screening studies at effective costs.

The lipid composition present in trophoblast cells at various gestational timepoints is currently well understood. While proteoliposomes have been previously developed and studied, the exemplary embodiments below provide the first cell-free technology towards placental trophoblast-inspired proteoliposomes.

FIG. 1A presents a schematic that is applicable for making the placental proteoliposomes (PPLs). In some embodiments. PPLs are developed using previously identified lipid composition (Table 1) representative of placental trophoblast cells. In some embodiments, one or more of three different transmembrane proteins important for understanding active transport across the placenta, including ATP-binding cassette proteins ABCB1 and ABCG2 and solute carrier SLC22A5 (Table 2) are incorporated into the PPLs.

TABLE 1
Lipid composition of placental proteoliposomes.

| Phospholipid | Percentage of Composition |
|---|---|
| PC | 50.7 ± 0.7 |
| PE | 23.3 ± 0.6 |
| PI | 7.0 ± 1.6 |
| PS | 9.1 ± 0.9 |
| SPH | 11.2 ± 0.7 |

TABLE 2
Transmembrane proteins for proteoliposome development.

| Protein | Function | Known substrates |
|---|---|---|
| ABCB1 | Drug extrusion pump | Antibiotics, antiemetic, |
| | | cardiac drugs, HIV |
| | | protease inhibitors |
| ABCG2 | Still relatively unknown, but key | Similar to ABCB1 |
| | player at the maternal-fetal interface | |
| | with placental expression at higher | |
| | level than any other organ | |
| Protein | Function | Known substrates |

In some embodiments, the PPLs are incorporated into extracellular matrix (ECM). Placental ECM and fluid further impact pharmaceutical and nutrient transport. The ECM orchestrates a complex environment of structural proteins, growth factors, cytokines, among other agents that influence the trophoblast cell's health and ability to migrate and invade the endometrium. Additionally, the cell's environment impacts how molecules interact with the cell membrane and transport proteins through both the physical and chemical cues present. Here, the developed PPLs are incorporated into ECM to form PPL-ECM. In some embodiments, the ECM or PPL-ECM are formed as droplets. In some embodiments, the ECM droplets contain ECM components such as structural proteins, growth factors, and cytokines (Table 3) that mimic the placental microenvironment.

TABLE 3
Placental extracellular matrix components.

| Structural proteins | Growth factors | Cytokines |
|---|---|---|
| Collagen IV, laminin, | Placental growth factor, | Tumor necrosis |
| fibronectin, heparan | vascular endothelial | factor (TNF)-alpha, |
| sulfate | growth factor (VEGF) | interleukin-1 |

### Example 2: Development of Placental Proteoliposome Compositions

This disclosure provides methods for forming one or more of the PPLs and PPL-ECM. The PPL is formed by employing a microfluidic mixing technology to develop the particles, which can be achieved via a NanoAssemblr R. Unlike more conventional techniques, including thin-film hydration and detergent based methods, the NanoAssemblrR: technique used here develops particles that are extremely reproducible, stable, and scale-up ready. This technique has previously been employed to formulate protein containing lipid vesicles. In some embodiments, to manufacture PPLs, an organic phase containing the placental lipid components in ethanol is mixed with an aqueous phase containing the placental protein in buffer. Mixing is facilitated through a microfluidic channel under laminar or turbulent flow.

In some embodiments, the PPLs are characterized for physiochemical properties to ensure formation. These characterization techniques include assessing the particle hydrodynamic diameter, polydispersity, and zeta potential via dynamic light scattering as well as performing cryo transmission electron microscopy (cryo-TEM) for structural analysis. The inclusion of the protein may be evident through cryo-TEM studies, but further characterization to ensure proper protein inclusion may be performed through differential scanning calorimetry (DSC), flow cytometry, and fourier-transform infrared spectroscopy studies (FTIR). Thermotropic behavior may be measured by DSC to observe differences in enthalpy changes when proteins are incorporated in the liposomes. Antibodies to tag the proteins incorporated in PPLs may be utilized to measure ABCB1. ABCG2, or SLC22A5 incorporation using flow cytometry. Spectrum peaks corresponding to the lipids and proteins are measured via FTIR.

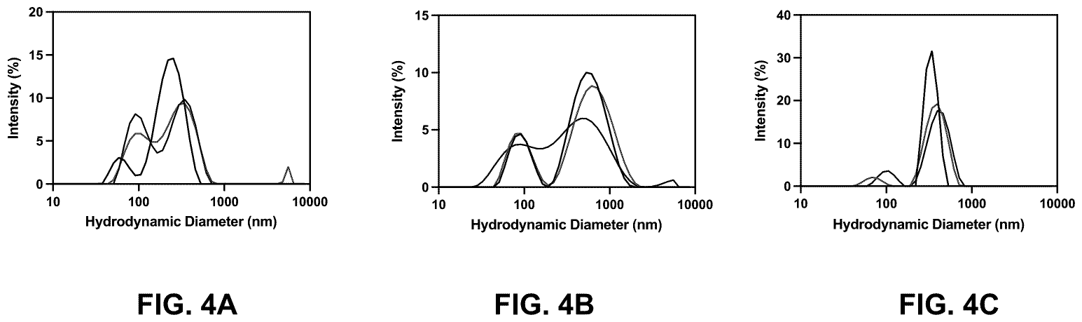

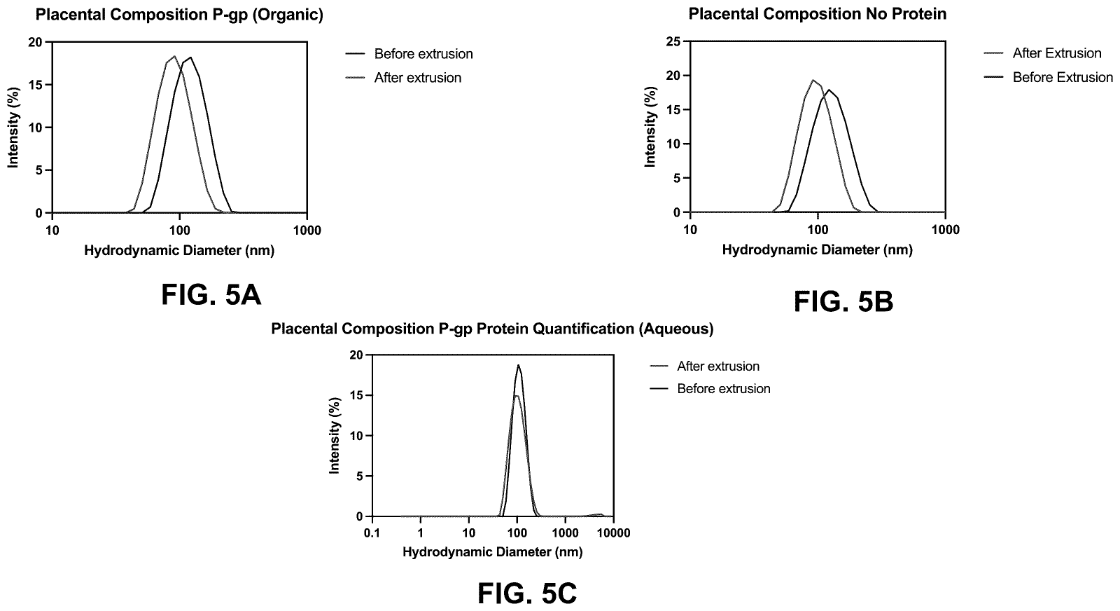

FIGS. 4A-4C present experimental results of PPLs using microfluidic mixing. FIGS. 5A-5C present experimental results of PPLs using extrusion. The results were obtained using an organic phase solution containing the one or more phospholipid in ethanol, with or without the one or more protein, mixed with an aqueous phase solution containing buffer, with or without the one or more placental protein.

In some embodiments, the PPLs are bioprinted into ECM droplets, with reference to FIG. 4. By using a bioprinter for this application, the droplets are developed in a high-throughput manner necessary for molecular transport studies in Example 3. In some embodiments, droplets are printed in 96 well plates to facilitate studies. In some embodiments, multiple printheads are used, allowed for a simple bioprinting workflow, where the ECM components are loaded into one printhead and the PPLs are loaded into another. Droplets are then extruded to form PPL loaded ECM models, referred herein as PPL-ECM. Due to the automation of this technique and high-throughput ability, bioprinting may be achieved using combinations of the components listed in Table 3. This enables identification of how each component influences transport properties.

### Example 3: Molecular Transport Assessment

In some aspects, this disclosure provides a method of using compositions for mimicking active transport in cells by assessing their capabilities. Specifically, models of both PPLs and PPL-ECM droplets, as shown in FIGS. 3A-3B, respectively, are used in transport studies to screen pharmaceutical and nutrient transport. Table 4 provides a list of some example pharmaceutical and nutrients for study with various molecular weights and hydrophobicity (log P), but many more compounds can be screened using both sets of models. Each model can be used to not only assess pharmaceutical and nutrient transport, but also a sequential study where after pharmaceutical interaction, nutrient transport is assessed. This enables a method to understand if the pharmaceutical is causing unwanted changes in nutrient transport by possibly impacting the protein, lipid, or ECM structure or altering the kinetics of the active transport.

TABLE 4
Example molecules for screening studies.

| | Molecular | | | |
|---|---|---|---|---|
| | Weight | | | |
| Molecule | (g/mol)^{a} | logP^{a} | Function^{a} | Relevance to women's health |
| Amphotericin B | 924.1 | 0.8 | Antifungal that binds to | Vulvo-vaginal candidiasis occurs |
| | | | ergosterol on the fungal | in 20% of pregnant women |
| | | | cell membrane, altering | |
| | | | permeability | |
| Carbamazepine | 236.27 | 2.45 | Tricyclic compounds with | Management of epilepsy during |
| | | | anticonvulsant and | pregnancy |
| | | | analgesic properties | |
| Sertaline | 306.2 | 5.51 | Selective serotonin | Management of depression, anxiety, |
| | | | reuptake inhibitor | and obsessive-compulsive disorder |
| | | | | during pregnancy. The rate of |
| | | | | pregnant women with depression |
| | | | | at the time of delivery increased |
| | | | | seven times from 2000 to 2015 |
| Folic acid | 441.4 | −2.5 | Water soluble vitamin | Essential vitamin during pregnancy |
| | | | | for prevention of major birth |
| | | | | defects, such as neural tube defects |
| Arginine | 174.2 | −4.2 | Amino acid, precursor | Amino acids, such as arginine, impact |
| | | | in nitric oxide and | fetal development and pregnancy |
| | | | polyamine synthesis | and help minimize complications, |
| | | | | such as intrauterine growth |
| | | | | restriction and preeclampsia |

^{a}Molecular weight, logP and function information was obtained from the National Library of Medicine, PubChem Database.

In some embodiments, for PPL transport studies, the molecule of interest is incubated with the PPLs with testing variables including incubation time, temperature, and buffer. In some embodiments, after incubation, the solution containing PPLs and any un-transported compounds is separated using an ultracentrifuge filter with a 100 kDa molecular weight cut-off. In some embodiments, PPLs remain in the filter compartment and the compound is passed through. In some embodiments, the filtered solution containing un-transported compound is quantified using UV-Vis or high-performance liquid chromatography (HPLC) depending on the detection ability of the molecule. In some embodiments, the collected PPLs are quantified pre- and post- disruption of the lipid structure using Triton X-100. This allows quantification of transport into the PPL.

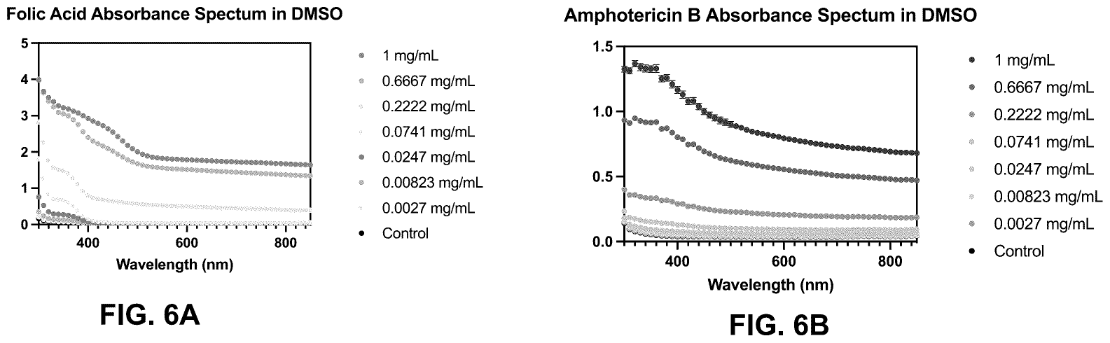

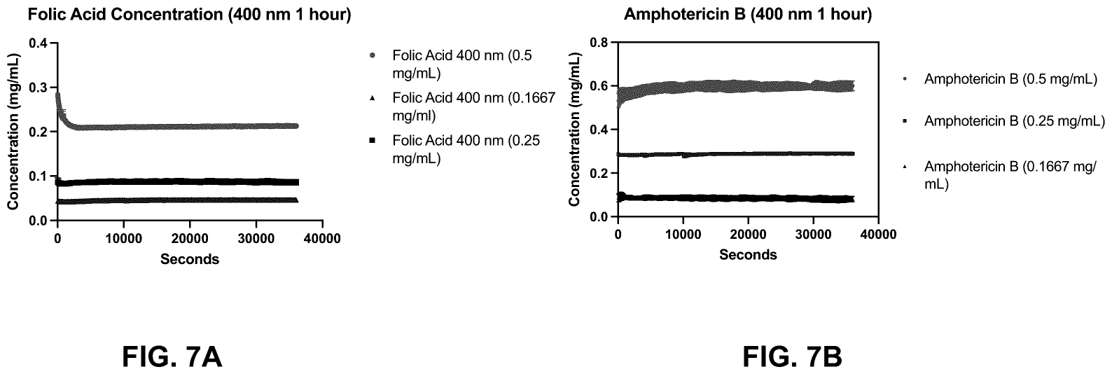

FIGS. 6A-6B provide measured absorbance spectra of the PPLs using Folic Acid and Amphotericin B as testing variables. FIGS. 7A-7B provide kinetic study results of the PPLS using Folic Acid and Amphotericin B as testing variables. The testing variables were measured using absorbance spectra above the UV range, e.g., 0-300 nm.

In some embodiments, similar incubation studies are performed with the PPL-ECM droplets. In some embodiments, after incubation, an additional step is required to degrade the structural protein. For example, if the structural protein studied is collagen, a solution of collagenase is added to the droplet. In some embodiments, an ultracentrifuge filter is used to collect the PPLs and the amount of compound transported into the PPL is measured. In some embodiments, controls of PPLs formed without the transport proteins are performed with both models. In some embodiments, transport across the PPL and PPL-ECM models are compared to transport across trophoblast cells.

By using this method, the impact of each component in the structure can be identified, a feature that is not readily available using a mammalian cell model. Ultimately, this is a facile method of screening pharmaceutical and nutrient transport and assessing the impact pharmaceuticals have on nutrient transport.

### Example 4: Kinetic Transport Assessment

By using this method, the formulated PPLs can be made on demand with better reproducibility than their cell derived counterparts. Additionally, they can be formulated to assess each component of the native particles, a feature that is not readily available using cell derived standards. The size of the biomimetic EVs can be tailored to meet the needs of the application from 50 nm to 1 μm.

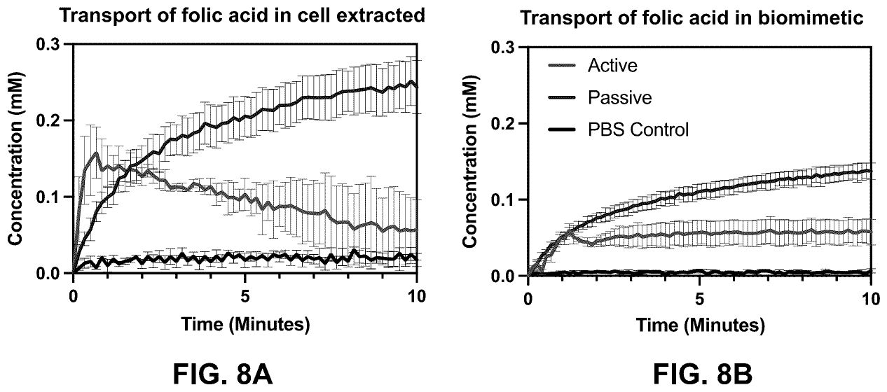

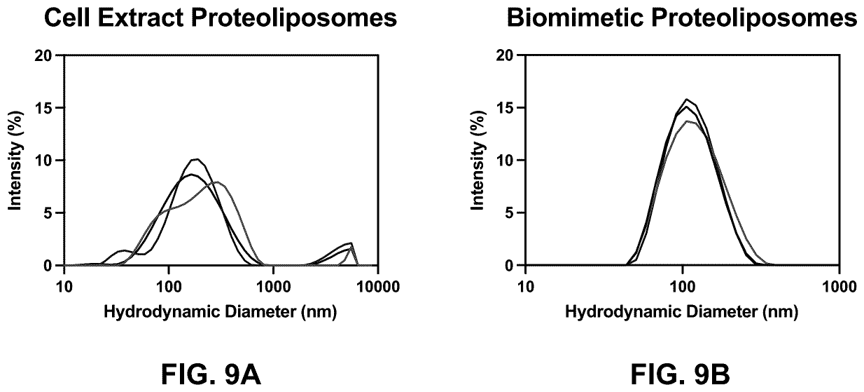

FIGS. 8A-8B provide kinetic study results of cell-extracted PPLs and biomimetic PPLs, respectively, using Folic Acid and Adenosine triphosphate (ATP) showing the activity of the proteins as testing variables, respectively. FIG. 8A depicts cell-derived vesicles extracted from placental trophoblast cells, and FIG. 8B depicts fully biomimetic proteoliposomes. i.e., the PPLs. In blue shows the transport through the bilayer without channel protein activity. i.e., just the Folic Acid. In red, the active transport through the bilayer is depicted from experimental results using ATP to drive the active transport. In black. PBS with no drug product is added as a negative control. The testing variables were measured using absorbance spectra above the UV range. e.g., 0-300 nm. The PPLs used in FIGS. 8A-8B comprise the lipid structure in Table 1 and ABCB1 channel proteins. FIGS. 9A-9B provide an example of hydrodynamic diameters of the PPLS, indicating comparable diameters to the cell-extracted PPLs to the biomimetic PPLs. The characterization techniques for obtaining results such as those in FIGS. 9A-9B, included assessing the particle hydrodynamic diameter, polydispersity, and zeta potential via dynamic light scattering as well as performing cryo transmission electron microscopy (cryo-TEM) for structural analysis.

### Example 5: Biomimetic Extracellular Vesicles/Nanoparticles

In some aspects, this disclosure provides compositions for representing reference standards or for critical quality attribute (CQA) assessment of pharmaceuticals for mimicking extracellular vesicles and other cell derived nanoparticles. The utility of the assay described is needed, as most EV therapeutics use stem cells which are cultured continuously, and EVs are extracted from the culture media. During this culturing the exposure to growth factors and other variables will affect the differentiation and thus the therapeutic efficacy of the derived therapeutics.

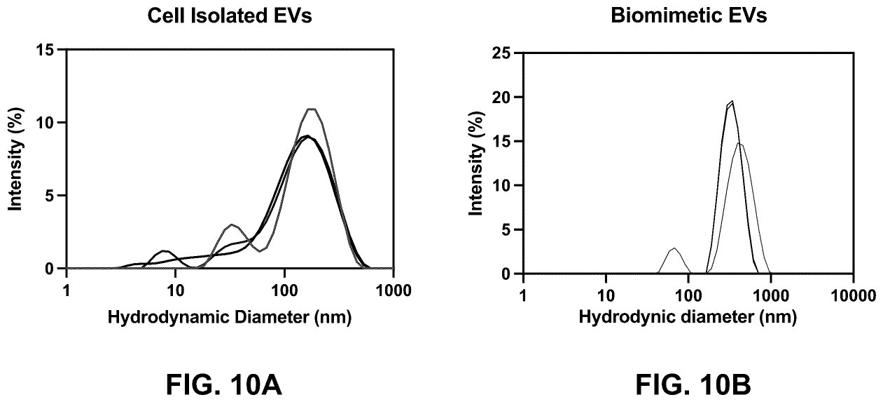

FIGS. 10A-10B present experimental results of isolated. i.e., cell-extracted. EVs in comparison to biomimetic EVs generated by the methods of this disclosure. The isolated EVs were extracted from placental trophoblast cells. The biomimetic EVs were produced by mixing phospholipids with proteins and nucleic acids, where the proteins comprise one or more of tetraspannins such as CD 9, 63, 81, or 82, integrins, Alix, TSG101, clathrin, Ubiquitin, HSP90, HSC90, HSP70, PD-L1, MHC, growth factors, lipoproteins, and polymerase proteins. The nucleic acids include at least miRNA such as one or more of miRNA 10, 21, 124, 125, 126, 130, and/or 132, mRNA encoding fluorescent protein, and/or other nucleic acids. The results were obtained by encapsulating nucleic acids in a lipid nanoparticle then extruding the lipid nanoparticle through a biomimetic phospholipid bilayer with or without protein. The characterization techniques included assessing the particle hydrodynamic diameter, polydispersity, and zeta potential via dynamic light scattering as well as performing cryo transmission electron microscopy (cryo-TEM) for structural analysis. The size of the biomimetic EVs can be tailored to meet the needs of the application from 50 nm to 1 μm.

### Example 6: Nucleic Acid Encapsulation Efficiency

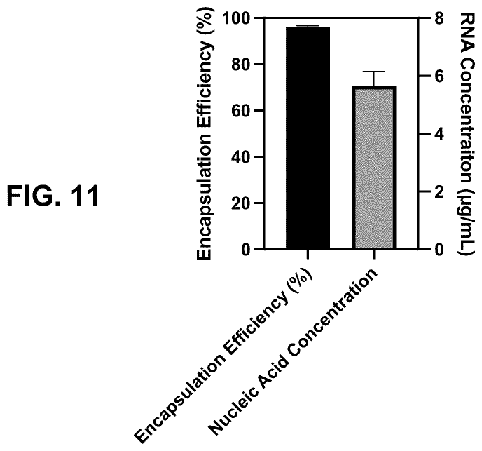

FIG. 11 presents experimental results of encapsulated nucleic acids, in particular nucleic acids that are encapsulated within biomimetic EVs generated by the methods of this disclosure. The biomimetic EVs were produced by mixing phospholipids with proteins and nucleic acids, where the proteins comprise one or more of tetraspannins such as CD 9, 63, 81, or 82, integrins, Alix, TSG101, clathrin, Ubiquitin, HSP90, HSC90, HSP70. PD-L1. MHC, growth factors, lipoproteins, and polymerase proteins. The nucleic acids include at least miRNA such as one or more of miRNA 10, 21, 124, 125, 126, 130, and/or 132. mRNA encoding fluorescent protein, and/or other nucleic acids. The results were obtained by encapsulating nucleic acids in a lipid nanoparticle then extruding the lipid nanoparticle through a biomimetic phospholipid bilayer with or without protein.

Encapsulation efficiency was evaluated by comparing the amount of nucleic acids were present outside of the biomimetic proteoliposomes relative to the amount inside the proteoliposomes. Concentrations of nucleic acids or proteins can be increased or decreased through concentration in particles or concentration of particles.

### Definitions

As used herein. “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%. 5%. 4%. 3%. 2%, or 1% from that value.

As used herein, a “cell” refers to a biological cell. Some non-limiting examples include: a prokaryotic cell, eukaryotic cell, a bacterial cell, an archaea cell, a cell of a single-cell eukaryotic organism, a protozoa cell, a cell from a plant, an algal cell, a fungal cell, a fungal protoplast cell, an animal cell, and the like. Sometimes a cell is not originating from a natural organism, e.g., a cell can be a synthetically made, sometimes termed an artificial cell.

As used herein, a “plurality” contains at least 2 members. In certain cases, a plurality may have at least 10, at least 100, at least 100, at least 10,000, at least 100,000, at least 106, at least 107, at least 108 or at least 109 or more members.

As used herein, when a quantitative characteristic (e.g., largest lateral dimension) is described as “in a range of,” when accompanied by a smaller value and a larger value, this refers to the quantitative characteristic having a value between the smaller value and the larger value or equal to the smaller value of the larger value.

As used herein, the characterizing term “uniform” in referencing a quantity (e.g., a distance, a thickness, a dimension (e.g., a largest lateral dimension)) refers to a variation in that quantity by no more than 10% more or less than the stated value or an average of that quantity (e.g., no more than 5% more or less, no more than 1% more or less, no more than 0.1% more or less than the stated value or an average of that quantity).

Although various features of the disclosure may be described in the context of a single embodiment, the features can also be provided separately or in any suitable combination. Conversely, although the disclosure may be described herein in the context of separate embodiments for clarity, various aspects and embodiments can be implemented in a single embodiment.

### INCORPORATION BY REFERENCE

All publications, patents, and patent applications mentioned in this specification are herein incorporated by reference to the same extent as if each individual publication, patent, or patent application was specifically and individually indicated to be incorporated by reference. Absent any indication otherwise, publications, patents, and patent applications mentioned in this specification are incorporated herein by reference in their entireties.

1. A proteoliposome comprising:
one or more phospholipid carrier and one or more protein embedded in the one or more phospholipid carrier;
wherein the one or more phospholipid carrier comprises a phospholipid composition with similar proportions of phospholipids as a naturally occurring cell type and a phospholipid concentration of about 1-50 mM; and
wherein the one or more protein comprises a protein composition with similar proportions of proteins as the naturally occurring cell type.

2. The proteoliposome of claim 1, wherein a ratio of the one or more phospholipid carrier to one or more protein is about 1:100.

3. The proteoliposome of claim 1, wherein the one or more phospholipid carrier comprises one or more of phosphatidylcholine (PC), phosphatidylethanolamine (PE), phosphatidylinositol (PI), phosphatidylserine (PS), and sphingomyelin (SPH) and, optionally, cholesterol.

4. (canceled)

5. The proteoliposome of claim 1, wherein the one or more protein comprises one or more transmembrane protein.

6. (canceled)

7. The proteoliposome of claim 1, further comprising extracellular matrix (ECM) to form a proteoliposome-ECM composition.

8. (canceled)

9. (canceled)

10. The proteoliposome of claim 1, further comprising one or more nucleic acid encapsulated within the proteoliposome.

11. (canceled)

12. (canceled)

13. (canceled)

14. The proteoliposome of claim 8, wherein the one or more nucleic acid is present at a concentration of 0-2 mg/mL.

15. (canceled)

16. The proteoliposome of claim 8, wherein the one or more nucleic acid comprises one or more of a chemical bond, nanoparticle, and a conjugation.

17. (canceled)

18. (canceled)

19. (canceled)

20. A method comprising:
(a) a first mixing of one or more phospholipid to form a phospholipid carrier solution; and
(b) a second mixing of the phospholipid carrier solution with a protein solution to produce one or more proteoliposome, wherein the protein solution comprises one or more protein.

21. The method of claim 19, wherein the second mixing comprises a microfluidics approach, wherein the microfluidics approach comprises flowing the phospholipid carrier solution and the protein solution through a microfluidic channel under at least one of laminar or turbulent flow.

22. The method of claim 19, wherein the second mixing comprises an extrusion approach, wherein the extrusion approach comprises extruding the phospholipid carrier solution and the protein solution through a porous membrane for a predetermined number of times.

23. The method of claim 19, wherein the second mixing comprises a combination of a microfluidic approach and an extrusion approach, wherein the microfluidic approach comprises flowing the phospholipid carrier solution and the protein solution through a microfluidic channel under at least one of laminar or turbulent flow, and wherein the extrusion approach comprises extruding the phospholipid carrier solution and the protein solution through a porous membrane a predetermined number of times.

24. The method of claim 19, wherein the phospholipid carrier solution comprises ethanol.

25. The method of claim 19, wherein the protein solution comprises in buffer.

26. The method of claim 19, wherein the one or more phospholipid comprises one or more of PC, PE, PI, PS, and SPH.

27. (canceled)

28. The method of claim 19, wherein the one or more protein comprises one or more transmembrane protein.

29. (canceled)

30. The method of claim 19, further comprising a third mixing of each of the one or more proteoliposome with ECM to form one or more proteoliposome-ECM.

31. (canceled)

32. (canceled)

33. The method of claim 29, wherein the third mixing comprises bioprinting.

34. (canceled)

35. (canceled)

36. (canceled)

37. A method for screening pharmaceuticals, comprising:
(a) incubating one or more of a placental proteoliposome (PPL) and a PPL-ECM with one or more molecule of interest to form an incubation product;
(b) filtering the incubation product to produce a filtered product; and
(c) quantifying the filtered product to assess transport of the one or more molecule of interest into one or more of the PPL and the PPL-ECM.

38. The method of claim 36, wherein the one or more molecule of interest comprises one or more of a pharmaceutical composition, a nutrient composition and a toxin.

39. (canceled)

40. (canceled)
